(19) United States
(12) Patent Application Publication
(10) Pub. No.: US 2005/0118126 A1
(43) Pub. Date: Jun. 2, 2005

(54) Cosmetic hair composition comprising at least one fixing polymer and at least one compound capable of swelling by the action of heat

(76) Inventors: Isabelle Rollat, Paris (FR); Jonathan Gawtrey, Boulogne (FR); Christine Condamine, Eragny S/Oise (FR)

Correspondence Address: Thomas L. Irving;FINNEGAN, HENDERSON, FARABOW,, GARRET & DUNNER, L.L.P., 1300 I Street, N.W., Washington, DC, 20005-3315, US

(21) Appl. No.: 10/969,924

(22) Filed: Oct. 22, 2004

(30) Foreign Application Priority Data
Oct. 24, 2003 (FR) 03 12486

(51) Int. Cl. A61K 7/06

(52) U.S. Cl. 424/70.1

(57) ABSTRACT

The present disclosure relates to cosmetic hair compositions, which comprise, in a cosmetically acceptable medium, at least one fixing polymer and at least one compound capable of swelling by the action of heat. The present disclosure also relates to a process for fixing and/or holding the hairstyle using the disclosed compositions.

The present disclosure relates to novel cosmetic hair compositions comprising at least one fixing polymer and at least one compound capable of swelling by the action of heat, such as microspheres with a thermoplastic envelope. The present disclosure also relates to a cosmetic process, for instance, a process for fixing and/or holding the hairstyle, and also to the cosmetic use of these compositions.

Haircare products for shaping and holding the hairstyle generally comprise one or more fixing polymers in alcoholic or aqueous solution. Fixing polymers ensure the formation of welds between the hairs. The fixing polymers generally used are film-forming polymers that are soluble or dispersible in water and in alcohols, such as copolymers of vinyl acetate and of crotonic acid, anionic or amphoteric acrylic resins, and polyurethanes.

The welds between the hairs should be strong enough to hold them. They should also be weak enough for the user to be able, by combing or brushing the hair, to break the welds without pulling on the scalp or damaging the hair, which is not always possible with the known hair holding and/or fixing compositions.

There is thus a need for hair products that can provide satisfactory hold of the hair, while at the same time allowing the user to subsequently style his or her hair without pulling on the scalp or damaging the hair.

Accordingly, the present disclosure provides a solution to this need.

For example, it has been discovered, surprisingly, that a cosmetic hair composition comprising, in a cosmetically acceptable medium, at least one fixing polymer and at least one compound capable of swelling by the action of heat can give the hair satisfactory and long-lasting fixing and that, by the effect of heat, the hair can then be easy to disentangle. Specifically, it has been found by the present inventors that, by the action of heat, the expansion of the compound capable of swelling results in embrittlement of the welds between the hairs, thus leading to a marked improvement in the disentangling of the hair.

Other aspects of the present disclosure will become apparent on reading the description and the examples that follow.

One aspect of the present disclosure is thus a cosmetic hair composition, comprising, in a cosmetically acceptable medium, at least one fixing polymer and at least one compound capable of swelling by the action of heat.

Another aspect of the present disclosure concerns a cosmetic hair process, for instance, a process for fixing and/or holding the hairstyle using the compositions according to the present disclosure.

Yet another aspect of the present disclosure is the cosmetic use of the compositions as disclosed herein.

For the purposes of the present disclosure, the term “fixing polymer” means any polymer that can give the hair a shape or that can maintain this shape. Fixing polymers that are suitable for use as disclosed herein include, for example, cationic, anionic, amphoteric and nonionic polymers and mixtures thereof.

Non-limiting examples of cationic fixing polymers that may be used as disclosed herein, include those chosen from polymers comprising primary, secondary, tertiary and/or quaternary amine groups forming part of the polymer chain or directly attached thereto, and having a number average molecular weight ranging from 500 to 5,000,000, such as from 1,000 to 3,000,000.

Among these polymers, non-limiting mention may be made of, for example, the following cationic polymers:
(1) homopolymers or copolymers derived from acrylic or methacrylic esters or amides with amine functional groups, and comprising at least one unit of the following formulae: 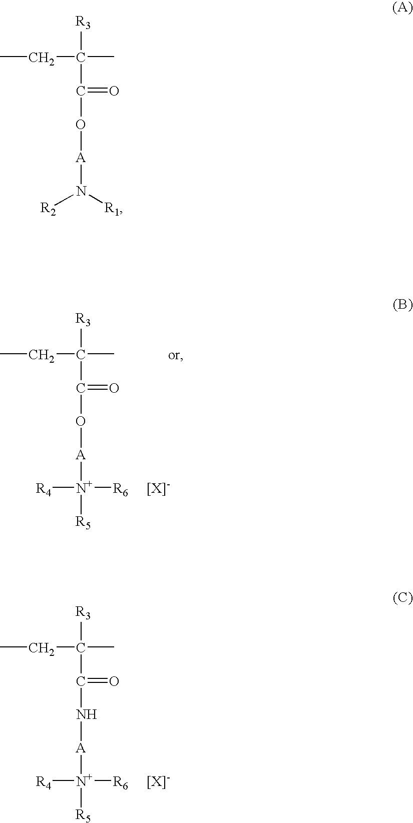
   wherein:

R₁ and R₂, which may be identical or different, are chosen from hydrogen atoms and alkyl groups having from 1 to 6 carbon atoms;

R₃ is chosen from a hydrogen atom and CH₃groups;

A is chosen from linear and branched alkyl groups comprising 1 to 6 carbon atoms and hydroxyalkyl groups comprising 1 to 4 carbon atoms;

R₄, R₅ and R₆, which may be identical or different, are chosen from alkyl groups having from 1 to 18 carbon atoms and benzyl groups;

X is chosen from methosulfate anions and halides such as chloride or bromide.

The copolymers of type (1) also comprise at least one comonomer unit that may be chosen from acrylamides, methacrylamides, diacetone acrylamides, acrylamides and methacrylamides substituted on the nitrogen with lower (C₁₋₄) alkyl groups; groups derived from acrylic or methacrylic acids or esters thereof, vinyllactams such as vinylpyrrolidone or vinylcaprolactam, and vinyl esters.

Further, among the copolymers of type (1), non-limiting mention may be made of:
copolymers of acrylamide and of dimethylaminoethyl methacrylate quaternized with dimethyl sulfate or with a dimethyl halide, such as the one sold under the name Hercofloc® by the company Hercules,
copolymers of acrylamide and of methacryloyloxyethyltrimethylammonium chloride, described, for example, in Patent Application EP-A-080 976 and sold under the name Bina Quat P 100 by the company Ciba Geigy,
copolymers of acrylamide and of methacryloyloxyethyltrimethylammonium methosulfate, such as the product sold under the name Reten by the company Hercules,
quaternized or non-quaternized vinylpyrrolidone/dialkylaminoalkyl acrylate or methacrylate copolymers, such as the products sold under the name “Gafquat®” by the company ISP, such as, for example, “Gafquat® 734” or “Gafquat® 755,” or alternatively the products known as “Copolymer® 845, 958 and 937.” These polymers are described in detail in French Patent Nos. 2,077,143 and 2,393,573,
fatty-chain polymers comprising a vinylpyrrolidone unit, such as the products sold under the name Styleze W20 and Styleze W10 by the company ISP,
dimethylaminoethyl methacrylate/vinylcaprolactam/vinylpyrrolidone terpolymers, such as the product sold under the name Gaffix® VC 713 by the company ISP, and
quaternized vinylpyrrolidone/dimethylaminopropylmethacrylamide copolymers, such as the product sold under the name “Gafquat® HS 100” by the company ISP;
(2) cationic polysaccharides, for instance comprising quaternary ammonium, such as those described in U.S. Pat. Nos. 3,589,578 and 4,031,307, such as guar gums comprising trialkylammonium cationic groups. Such products are sold for example, under the trade names Jaguar C13S, Jaguar C15 and Jaguar C17 by the company Meyhall;
(3) quaternary copolymers of vinylpyrrolidone and of vinylimidazole;
(4) chitosans or salts thereof; the salts that can be used include, for example, chitosan acetate, lactate, glutamate, gluconate or pyrrolidonecarboxylate.

Among these compounds, non-limiting mention may be made of chitosan having a degree of deacetylation of 90.5% by weight, sold under the name Kytan Brut Standard by the company Aber Technologies, and chitosan pyrrolidonecarboxylate sold under the name Kytamer® PC by the company Amerchol.
(5) cationic cellulose derivatives such as copolymers of cellulose or of cellulose derivatives grafted with a water-soluble monomer comprising a quaternary ammonium, and disclosed, for example, in U.S. Pat. No.4,131,576, such as hydroxyalkylcelluloses, for instance hydroxymethyl-, hydroxyethyl- or hydroxypropylcelluloses grafted for example, with a methacryloyloxyethyltrimethylammonium, methacrylamidopropyltrimethylammonium or dimethyldiallylammonium salt. The products sold corresponding to this definition include, for instance, the products sold under the name “Celquat L 200” and “Celquat H 100” by the company National Starch.

The anionic fixing polymers that may be used according to the present disclosure are polymers comprising groups derived from carboxylic acid, sulfonic acid or phosphoric acid and having a number average molecular weight ranging from 500 to 5,000,000.

The carboxylic groups are provided by unsaturated monocarboxylic or dicarboxylic acid monomers such as those of formula: 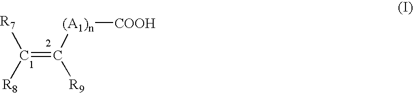
wherein

n is an integer ranging from 0 to 10;

A₁ is a methylene group, optionally connected to the carbon atom of the unsaturated group, or to the neighboring methylene group, when n is greater than 1, via a hetero atom such as oxygen or sulfur;

R₇ is chosen from a hydrogen atom and phenyl and benzyl groups;

R₈ is chosen from a hydrogen atom and lower alkyl and carboxyl groups; and

R₉ is chosen from a hydrogen atom, lower alkyl groups, —CH₂—COOH groups, phenyl groups and benzyl groups.

In one embodiment of the abovementioned formula, a lower alkyl group denotes a group having 1 to 4 carbon atoms, such as methyl and ethyl.

Among the anionic fixing polymers comprising carboxylic groups that may be used according to the present disclosure, non-limiting mention may be made of:
A) acrylic or methacrylic acid homo- or copolymers, or salts thereof, for example the products sold under the names Versicol® E or K by the company Allied Colloid and Ultrahold® by the company BASF, the copolymers of acrylic acid and of acrylamide sold in the form of their sodium salts under the names Reten 421, 423 or 425 by the company Hercules, the sodium salts of polyhydroxycarboxylic acids.
B) copolymers of acrylic or methacrylic acid with a monoethylenic monomer such as ethylene, styrene, vinyl esters, acrylic or methacrylic acid esters, optionally grafted onto a polyalkylene glycol such as polyethylene glycol and optionally crosslinked. Such polymers are described for instance in French Patent No. 1,222,944 and German Patent Application No. 2,330,956, the copolymers of this type comprising an optionally N-alkylated and/or hydroxyalkylated acrylamide unit in their chain as described for example in Luxembourg Patent Application Nos. 75370 and 75371, or sold under the name Quadramer by the company American Cyanamid. Non-limiting mention may also be made of copolymers of acrylic acid and of C₁-C₄ alkyl methacrylate, and terpolymers of vinylpyrrolidone, of acrylic acid and of methacrylate of C₁-C₂₀ alkyl, for example of lauryl, such as the product sold by the company ISP under the name Acrylidone® LM and methacrylic acid/ethyl acrylate/tert-butyl acrylate terpolymers such as the product sold under the name Luvimer® 100 P by the company BASF.

Non-limiting mention may also be made of methacrylic acid/acrylic acid/ethyl acrylate/methyl methacrylate copolymers as an aqueous dispersion, sold under the name Amerhold® DR 25 by the company Amerchol.
C) crotonic acid copolymers, such as those comprising vinyl acetate or propionate units in their chain and optionally other monomers such as allylic esters or methallylic esters, vinyl ether or vinyl ester of a linear or branched saturated carboxylic acid with a long hydrocarbon chain such as those comprising at least 5 carbon atoms, it being possible for these polymers optionally to be grafted or crosslinked, or alternatively another vinyl, allylic or methallylic ester monomer of an α- or β-cyclic carboxylic acid. Such polymers are described, inter alia, in French Patent Nos. 1,222,944, 1,580,545, 2,265,782, 2,265,781, 1,564,110 and 2,439,798. Commercial products falling into this class are the resins 28-29-30, 26-13-14 and 28-13-10 sold by the company National Starch.
D) copolymers derived from C₄-C₈ monounsaturated carboxylic acids or anhydrides chosen from:
copolymers comprising (i) at least one of maleic, fumaric or itaconic acids or anhydrides and (ii) at least one monomer chosen from vinyl esters, vinyl ethers, vinyl halides, phenylvinyl derivatives, acrylic acid and its esters, the anhydride functional groups of these copolymers optionally being monoesterified or monoamidated. Such polymers are described, for example, in U.S. Pat. Nos. 2,047,398, 2,723,248, and 2,102,113, and Great Britain Patent No. GB 839,805, for instance, those sold under the names Gantrez® AN or ES by the company ISP,
copolymers comprising (i) at least one of maleic, citraconic or itaconic anhydride unit and (ii) at least one monomer chosen from allylic or methallylic esters optionally comprising at least one group chosen from acrylamide, methacrylamide, α-olefin, acrylic or methacrylic ester, acrylic or methacrylic acid or vinylpyrrolidone groups in their chain, the anhydride functional groups of these copolymers optionally being monoesterified or monoamidated.

These polymers are described, for example, in French Patent Nos. FR 2,350,384 and FR 2,357,241.
E) polyacrylamides comprising carboxylate groups.

The homopolymers and copolymers comprising sulfonic groups are polymers comprising vinylsulfonic, styrenesulfonic, naphthalenesulfonic or acrylamido-alkylsulfonic units.

These polymers can be chosen, for example, from:
polyvinylsulfonic acid salts having a molecular weight ranging from 1,000 to 100,000, as well as the copolymers with an unsaturated comonomer such as acrylic or methacrylic acids and their esters, as well as acrylamide or its derivatives, vinyl ethers and vinylpyrrolidone;
polystyrenesulfonic acid salts such as the sodium salts that are sold under the names Flexan® 500 and Flexan® 130 by National Starch. These compounds are described in French Patent No. FR 2,198,719;
polyacrylamidesulfonic acid salts, such as those mentioned in U.S. Pat. No. 4,128,631, and for instance polyacrylamidoethylpropanesulfonic acid sold under the name Cosmedia Polymer HSP 1180 by Henkel.

As another example of anionic fixing polymers that can be used as disclosed herein, non-limiting mention may be made of the branched block anionic polymer sold under the name Fixate G100 by the company Noveon.

According to the present disclosure, the anionic fixing polymers can be chosen from, for example, acrylic acid copolymers, such as the acrylic acid/ethyl acrylate/N-tert-butylacrylamide terpolymers sold, for instance, under the name Ultrahold® Strong by the company BASF, copolymers derived from crotonic acid, such as vinyl acetate/vinyl tert-butylbenzoate/crotonic acid terpolymers and the crotonic acid/vinyl acetate/vinyl neododecanoate terpolymers, such as those sold under the name Resin 28-29-30 by the company National Starch, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold under the name Gantrez® by the company ISP, the copolymers of methacrylic acid and of methyl methacrylate sold under the name Eudragit® L by the company Rohm Pharma, the copolymers of methacrylic acid and of ethyl acrylate sold under the name Luvimer® MAEX or MAE by the company BASF, the vinyl acetate/crotonic acid copolymers sold under the name Luviset CA 66 by the company BASF, the vinyl acetate/crotonic acid copolymers grafted with polyethylene glycol sold under the name Aristoflex A® by the company BASF, and the polymer sold under the name Fixate G100 by the company Noveon.

Further among the anionic fixing polymers discussed above, non-limiting mention may be made, for example, of methyl vinyl ether/monoesterified maleic anhydride copolymers sold under the name Gantrez® ES 425 by the company ISP, the acrylic acid/ethyl acrylate/N-tert-butylacrylamide terpolymers sold under the name UUltrahold® Strong by the company BASF, the copolymers of methacrylic acid and of methyl methacrylate sold under the name Eudragit® L by the company Rohm Pharma, the vinyl acetate/vinyl tert-butylbenzoate/crotonic acid terpolymers and the crotonic acid/vinyl acetate/vinyl neododecanoate terpolymers sold under the name Resin 28-29-30 by the company National Starch, the copolymers of methacrylic acid and of ethyl acrylate sold under the name Luvimer® MAEX or MAE by the company BASF, the vinylpyrrolidone/acrylic acid/lauryl methacrylate terpolymers sold under the name Acrylidone LM by the company ISP, and the polymer sold under the name Fixate G100 by the company Noveon.

The amphoteric fixing polymers that can be used in accordance with the present disclosure can be chosen from polymers comprising units B and C distributed randomly in the polymer chain, wherein B is a unit derived from a monomer comprising at least one basic nitrogen atom and C is a unit derived from an acid monomer comprising at least one carboxylic or sulfonic group, or alternatively B and C can be groups derived from carboxybetaine or sulfobetaine zwitterionic monomers;

B and C can also be chosen from cationic polymer chains comprising primary, secondary, tertiary or quaternary amine groups, in which at least one of the amine groups bears a carboxylic or sulfonic group connected via a hydrocarbon group, or alternatively B and C form part of a chain of a polymer comprising an α,β-dicarboxylic ethylene unit in which one of the carboxylic groups has been made to react with a polyamine comprising one or more primary or secondary amine groups.

Among the amphoteric fixing polymers corresponding to the definition given above, non-limiting mention may be made of those chosen from:
(1) copolymers having acidic vinyl and basic vinyl units, such as those resulting from the copolymerization of a monomer derived from a vinyl compound bearing a carboxylic group such as, for instance, acrylic acid, methacrylic acid, maleic acid, α-chloroacrylic acid, and a basic monomer derived from a substituted vinyl compound comprising at least one basic atom, such as, for example, dialkylaminoalkyl methacrylate and acrylate, dialkylaminoalkylmethacrylamides and -acrylamides. Such compounds are described in U.S. Pat. No. 3,836,537.
(2) polymers comprising units derived from:
   a) at least one monomer chosen from acrylamides and methacrylamides substituted on the nitrogen with an alkyl group,
   b) at least one acidic comonomer comprising at least one reactive carboxylic group, and
   c) at least one basic comonomer such as esters comprising primary, secondary, tertiary and quaternary amine substituents of acrylic and methacrylic acids and the product of quaternization of dimethylaminoethyl methacrylate with dimethyl or diethyl sulfate.

Among the N-substituted acrylamides or methacrylamides that may be used according to the present disclosure include compounds in which the alkyl groups comprise from 2 to 12 carbon atoms, such as N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers may be chosen from, for example from acrylic acid, methacrylic acid, crotonic acid, itaconic acid, maleic acid and fumaric acid and alkyl monoesters, having 1 to 4 carbon atoms, of maleic or fumaric acids or anhydrides.

In one embodiment of the present disclosure, the basic comonomers are chosen from aminoethyl, butylaminoethyl, N,N′-dimethylaminoethyl and N-tert-butylaminoethyl methacrylates.

The copolymers whose CTFA (4th edition, 1991) name is octylacrylamide/acrylates/butylaminoethyl methacrylate copolymer, such as the products sold under the name Amphomer® or Lovocryl® 47 by the company National Starch, for example, may be used.
(3) crosslinked and acylated polyamino amides partially or totally derived from polyamino amides of general formula:
   CO—R₁₀—CO—Z (II)
   wherein R₁₀ is chosen from divalent groups derived from a saturated dicarboxylic acid, a mono- or dicarboxylic aliphatic acid comprising an ethylenic double bond, an ester of a lower alkanol, having 1 to 6 carbon atoms, of these acids, or a group derived from the addition of any one of these acids to a bis(primary) or bis(secondary) amine, and Z is chosen from groups derived from a bis(primary), mono- or bis(secondary) polyalkylene-polyamine and, for example, may be chosen from:
a) in an amount ranging from 60 to 100 mol %, the group:
   —NH(CH₂)ₓ—NHₚ (IV)
   where x=2 and p=2 or 3, or alternatively x=3 and p=2 this group being derived from diethylenetriamine, from triethylenetetraamine or from dipropylenetriamine;
b) in an amount ranging from 0 to 40 mol %, the group (IV) above in which x=2 and p=1 and which is derived from ethylenediamine, or the group derived from piperazine: 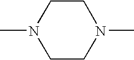
c) in an amount ranging from 0 to 20 mol %, the —NH(CH₂)₆—NH— group being derived from hexamethylenediamine, the polyamino amides being crosslinked by addition reaction of a difunctional crosslinking agent chosen from epihalohydrins, diepoxides, dianhydrides and bis-unsaturated derivatives, using from 0.025 to 0.35 mol of crosslinking agent per amine group of the polyamino amide and acylated by the action of acrylic acid, chloroacetic acid or an alkane sultone, or salts thereof.

The saturated carboxylic acids may be chosen from, for example, acids having 6 to 10 carbon atoms, such as adipic acid, 2,2,4-trimethyladipic acid and 2,4,4-trimethyladipic acid, terephthalic acid, acids containing an ethylenic double bond such as, for example, acrylic acid, methacrylic acid and itaconic acid.

The alkane sultones used in the acylation may be chosen from, for instance, propane sultone or butane sultone, the salts of the acylating agents may be chosen from, for example, sodium or potassium salts.
(4) polymers comprising zwitterionic units of formula: 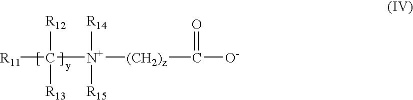
   wherein R₁₁ is chosen from a polymerizable unsaturated group such as an acrylate, methacrylate, acrylamide or methacrylamide group, y and z are integers ranging from 1 to 3, R₁₂ and R₁₃ are chosen from hydrogen atoms, a methyl, ethyl and propyl groups, R₁₄ and R₁₅ are chosen from hydrogen atoms and alkyl groups such that the sum of the carbon atoms in R₁₄ and R₁₅ does not exceed 10.

The polymers comprising such units can also comprise units derived from nonzwitterionic monomers such as dimethyl- or diethylaminoethyl acrylate or methacrylate or alkyl acrylates or methacrylates, acrylamides or methacrylamides or vinyl acetate.

By way of example, non-limiting mention may be made of the copolymers of methyl methacrylate/methyl dimethylcarboxymethylammonioethyl methacrylate such as the product sold under the name Diaformer Z301 by the company Sandoz.
(5) polymers derived from chitosan comprising monomer units of the following formulae: 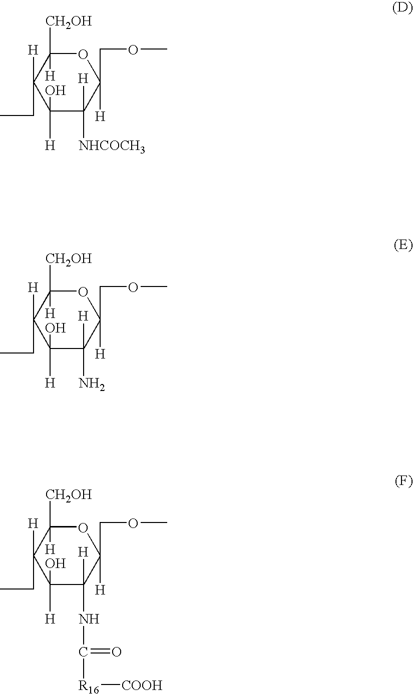
   wherein the unit (D) is present in an amount ranging from 0 to 30%, the unit (E) is present in an amount ranging from 5 to 50%, and the unit (F) is present in an amount ranging from 30% to 90%, it being understood that, in unit (F), R₁₆ is a group of formula: 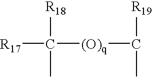
   wherein, when q is equal to zero, R₁₇, R₁₈ and R₁₉, which may be identical or different, are chosen from hydrogen atoms, methyl, hydroxyl, acetoxy and amino radicals, monoalkylamine radicals, and dialkylamine radicals that are optionally interrupted by at least one nitrogen atom and/or optionally substituted with at least one radical chosen from amine, hydroxyl, carboxyl, alkylthio and sulfonic radicals, alkylthio radicals in which the alkyl group bears an amino radical,

with the proviso that when q is equal to zero, at least one of the groups R₁₇, R₁₈ and R₁₉ is a hydrogen atom;

and wherein, when q is equal to 1, R₁₇, R₁₈ and R₁₉ are hydrogen atoms, as well as the salts formed by these compounds with bases or acids.
(6) polymers of formula (V) that are described, for example, in French Patent No. 1,400,366: 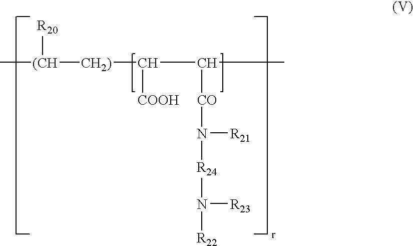
   wherein

R₂₀ is chosen from a hydrogen atom, CH₃O, CH₃CH₂O and phenyl groups, R₂₁ is chosen from a hydrogen atom and lower alkyl groups, such as methyl or ethyl,

R₂₂ is chosen from a hydrogen atom or C₁₋₆ lower alkyl groups, such as methyl or ethyl,

R₂₃ is chosen from C₁₋₆ lower alkyl groups, such as methyl or ethyl, and groups corresponding to the formula: —R₂₄—N(R₂₂)₂, wherein R₂₄ is chosen from —CH₂—CH₂—, —CH₂—CH₂—CH₂— and —CH₂—CH(CH₃)— groups, R₂₂ having the meaning given above, and r is an integer greater than 1.
(7) polymers derived from the N-carboxyalkylation of chitosan, such as N-carboxymethylchitosan or N-carboxybutylchitosan sold under the name “Evalsan” by the company Jan Dekker.
(8) amphoteric polymers of the type —D—X—D—X— chosen from:
a) polymers derived from the reaction of chloroacetic acid or sodium chloroacetate with compounds comprising at least one unit of formula:
   —D—X—D—X—D— (VI)
   where D is a group 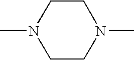
   and X is chosen from E and E′, wherein E and E′, which may be identical or different, are divalent groups chosen from straight and branched chain alkylene groups comprising up to 7 carbon atoms in the main chain, which is unsubstituted or substituted with hydroxyl groups and which can comprise oxygen, nitrogen and sulfur atoms, and 1 to 3 aromatic and/or heterocyclic rings; wherein the oxygen, nitrogen and sulfur atoms are present in the form of ether, thioether, sulfoxide, sulfone, sulfonium, alkylamine, alkenylamine, hydroxyl, benzylamine, amine oxide, quaternary ammonium, amide, imide, alcohol, ester and/or urethane groups.
b) polymers of formula:
   —D—X—D—X— (VI′)
   wherein D is a group 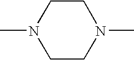
   and X is chosen from E and E′, with the proviso that X is at least once E′; wherein E has the meaning given above and E′ is a divalent group chosen from straight and branched chain alkylene groups comprising up to 7 carbon atoms in the main chain, which is unsubstituted or substituted with at least one hydroxyl group and comprising at least one nitrogen atom, wherein the nitrogen atom is substituted with an alkyl chain that is optionally interrupted by an oxygen atom and wherein the alkyl chain comprises at least one carboxyl functional group or at least one hydroxyl functional group and betainized by reaction with chloroacetic acid or sodium chloroacetate.
(9) (C₁-C₅)alkyl vinyl ether/maleic anhydride copolymers partially modified by semiamidation with an N,N-dialkylaminoalkylamine, such as N,N-dimethylaminopropylamine, or by semiesterification with an N,N-dialkylaminoalkanol. These copolymers can also comprise other vinyl comonomers such as vinylcaprolactam.

Among the amphoteric fixing polymers described above, non-limiting mention may be made of those of type (3), such as the copolymers whose CTFA name is octylacrylamide/acrylates/butylaminoethyl methacrylate copolymer, such as the products sold under the names Amphomer®, Amphomer® LV71 or Lovocryl® 47 by the company National Starch and those of type (4) such as the copolymers of methyl methacrylate/methyl dimethylcarboxymethylammonioethyl methacrylate, sold, for example, under the name Diaformer® Z301 by the company Sandoz.

The nonionic fixing polymers that may be used as disclosed herein are chosen, for example, from:
polyalkyloxazolines;
vinyl acetate homopolymers;
vinyl acetate copolymers, for instance copolymers of vinyl acetate and of acrylic ester; copolymers of vinyl acetate and of ethylene; copolymers of vinyl acetate and of maleic ester, for example of dibutyl maleate;
homopolymers and copolymers of acrylic esters, for instance copolymers of alkyl acrylates and of alkyl methacrylates, such as the products sold by the company Rohm & Haas under the names Primal® AC-261 K and Eudragit® NE 30 D, by the company BASF under the name 8845, or by the company Hoechst under the name Appretan® N9212;
copolymers of acrylonitrile and of a nonionic monomer chosen, for example, from butadiene and alkyl (meth)acrylates; non-limiting mention may be made of the products sold under the name CJ 0601 B by the company Rohm & Haas;
styrene homopolymers;
styrene copolymers, for instance copolymers of styrene and of an alkyl (meth)acrylate, such as the products Mowilith® LDM 6911, Mowilith® DM 611 and Mowilith® LDM 6070 sold by the company Hoechst, and the products Rhodopas® SD 215 and Rhodopas® DS 910 sold by the company Rhône-Poulenc; copolymers of styrene, of alkyl methacrylate and of alkyl acrylate; copolymers of styrene and of butadiene; or copolymers of styrene, of butadiene and of vinylpyridine;
polyamides;
vinyllactam homopolymers other than vinylpyrrolidone homopolymers, such as the polyvinylcaprolactam sold under the name Luviskol® Plus by the company BASF; and
vinyllactam copolymers such as a poly(vinylpyrrolidone/vinyllactam) copolymer sold under the trade name Luvitec® VPC 55K65W by the company BASF, poly(vinylpyrrolidone/vinyl acetate) copolymers, such as those sold under the name PVPVA® S630L by the company ISP, Luviskol® VA 73, VA 64, VA 55, VA 37 and VA 28 by the company BASF; and poly(vinylpyrrolidone/vinyl acetate/vinyl propionate) terpolymers, for instance the product sold under the name Luviskol® VAP 343 by the company BASF.

In one embodiment of the present disclosure, the alkyl groups of the nonionic polymers mentioned above may comprise from 1 to 6 carbon atoms.

According to the present disclosure, it is also possible to use fixing polymers of grafted silicone type comprising a polysiloxane portion and a portion comprising a nonsilicone organic chain, wherein one of the two portions constitutes the main chain of the polymer, and the other being grafted onto the said main chain. Such polymers are described, for example, in the patent applications: EP-A-0 412 704, EP-A-0 412 707, EP-A-0 640 105 and WO 95/00578, EP-A-0 582 152 and WO 93/23009 and in U.S. Pat. Nos. 4,693,935, 4,728,571 and 4,972,037.

The grafted silicone polymers may be chosen from amphoteric, anionic and nonionic polymers. In one embodiment of the present disclosure, the grafted silicone polymers are chosen from anionic and nonionic polymers.

Such polymers are, for example, copolymers that can be obtained by free radical polymerization from the monomer mixture formed from:
a) from 50 to 90% by weight of tert-butyl acrylate;
b) from 0 to 40% by weight of acrylic acid;
c) from 5 to 40% by weight of a silicone macromer of formula: 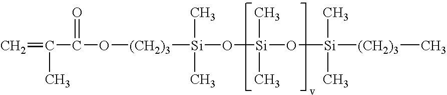
   in which v is a number from 5 to 700; the weight percentages being calculated relative to the total weight of the monomers.

Other non-limiting examples of grafted silicone polymers include, for example, polydimethylsiloxanes (PDMSs) onto which are grafted, via a thiopropylene-type connecting chain, mixed polymer units of the poly(meth)acrylic acid type and of the polyalkyl (meth)acrylate type; and polydimethylsiloxanes (PDMSs) onto which are grafted, via a thiopropylene-type connecting chain, polymer units of the polyisobutyl (meth)acrylate type. Non-limiting mention may also be made of the product Luviflex® Silk, sold by the company BASF.

Functionalized or non-functionalized, silicone or non-silicone, cationic, nonionic, anionic or amphoteric polyurethanes or mixtures thereof may also be used as fixing polymers.

Non-limiting examples of polyurethanes that may be used as disclosed herein are those described in European Patent Nos. EP 0 751 162, EP 0 637 600, and EP 0 648 485 and French Patent No. FR 2,743,297, as well as the Patent Nos. EP 0 656 021, WO 94/03510, and EP 0 619 111.

Among the polyurethanes that may be used as disclosed herein, non-limiting mention may be made of the products sold under the names Luviset Pur® and Luviset® Si-Pur by the company BASF.

In the compositions of the present disclosure, the at least one fixing polymer is present in an amount ranging from 0.1% to 20%, for instance, from 0.2% to 15%, such as from 0.5% to 10% by weight, relative to the total weight of the composition.

As indicated above, the compositions according to the present disclosure comprise at least one compound capable of swelling by the action of heat. As used herein, “swelling” and “expanding” are interchangeable.

This compound can be, for example, a compound that reacts, under the action of heat, to release a gas that is trapped within the matrix of the deposit.

The compound capable of swelling upon heating may also be in the form of heat-expandable particles.

As used herein, the expression “heat-expandable particles” indicates, for example, particles capable of becoming deformed and of expanding on heating. The particles within the meaning of the present disclosure may also be non-expanded heat-deformable particles. They are distinguished in this respect from expanded particles, which are, for instance, no longer subject to deformation under the action of heat in the manner, for example, of the polyvinylidene/acrylonitrile particles sold under the generic name “Expancel®” by the company Akzo Nobel, for example, under the references “Expancel® WE” or “DE”.

The particles used in the compositions according to the present disclosure are capable of expanding under the action of a temperature greater than or equal to 45° C., for instance, greater than or equal to 50° C., such as greater than or equal to 60° C. and greater than or equal to 70° C. For example, it may be a temperature of greater than or equal to 80° C., for instance, greater than or equal to 85° C., such as greater than or equal to 90° C., and for example, ranging up to 190-200° C.

In one embodiment of the present disclosure, these particles are not sensitive to the presence of water.

In another embodiment, the particles used in the present disclosure are thermoplastic. The term “thermoplastic” means particles that are capable of becoming deformed under the action of heat and of keeping their new shape, including after cooling, such as to room temperature.

The particles used in the present disclosure can be hollow particles comprising a continuous envelope and at least one cavity. The envelope of the particles is flexible to allow mechanical deformation. It generally comprises at least one polymer, a homopolymer or copolymer, formed from ethylenically unsaturated monomers. Examples of such particles are described, for example, in European Patent Nos. EP-A-56219, EP-A-348 372, EP-A-486 080, EP-A-320 473, EP-A-112 807 and U.S. Pat. No. 3,615,972.

The monomers used may be for example, methacrylic or acrylic acid esters, such as methyl acrylate and methacrylate, vinylidene chloride, acrylonitrile, styrene and derivatives thereof, butadiene and derivatives thereof, and mixtures thereof.

As non-limiting examples of the polymers of which the envelope of the particles used as disclosed herein may be composed, mention may be made of, for example, polymers comprising at least one monomeric unit chosen from methyl acrylate and methacrylate derivatives, polymers comprising at least one monomeric unit chosen from acrylonitrile derivatives, polymers comprising at least one monomeric unit chosen from acrylonitrile and methyl methacrylate derivatives, polymers comprising at least one monomeric unit chosen from styrene and acrylonitrile derivatives, polymers-comprising at least one monomeric unit chosen from vinylidene chloride and acrylonitrile derivatives, and polymers comprising at least at least one monomeric unit chosen from vinylidene chloride and vinyl chloride derivatives. For instance, the polymer may be chosen from vinylidene chloride/acrylonitrile/methyl methacrylate polymers, acrylonitrile/methyl methacrylate polymers and acrylonitrile homopolymers.

The particles generally comprise within at least one cavity at least one compound capable of exhibiting, in response to heating to a temperature ranging from 45° C. to 200° C. and at substantially constant pressure, a significant increase in its volume relative to its volume at room temperature.

As used herein, the expression “significant increase in its volume” means an increase by at least a factor of 30, for instance by at least a factor of 40, such as by at least a factor of 50 of the occupied volume.

In general, the compound comprised within the cavity may be, at room temperature, a gaseous compound or a liquid compound with a vaporization temperature ranging from 45° C. to 200° C., for instance ranging from 80° C. to 200° C., and such as greater than or equal to 100° C.

In one embodiment of the present disclosure, the compound is in the gaseous form in the particle and becomes dilated under the effect of heat. Among the compounds in gaseous form, non-limiting mention may be made of air, nitrogen, hydrocarbons comprising from 1 to 4 carbon atoms, for instance butane or isobutane, and mixtures thereof.

In another embodiment of the present disclosure, the compound comprised in the cavity is a liquid compound as defined above. Among these compounds, non-limiting mention may be made of hydrocarbons, for instance, comprising from 5 to 15, for example, from 5 to 12 and such as from 5 to 10 carbon atoms. It may be, for instance, a compound chosen from n-pentane, isopentane and neopentane.

The expansion temperature of the particle depends both on the nature of the compound present in its cavity and on the polymer forming its envelope, and it may range for example, from 45 to 200°C., and may for instance be greater than or equal to 80° C., such as greater than or equal to 100°°C.

The particles used in the compositions according to the present disclosure can be dry or hydrated.

These particles can have various shapes, for example, they can be of globular or even spherical shape, or may also be elongated.

According to one embodiment of the present disclosure, the non-expanded particles as disclosed herein are spherical and have a particle size, expressed as the weight-average “effective” diameter D[0.5], ranging from 0.5 μm to 200 μm, for instance from 1 μm to 100 μm, such as from 4 μm to 50 μm and from 5 μm to 40 μm.

According to another embodiment of the present disclosure, the particles used in the compositions as disclosed herein have a fiber. The term “fiber” as used herein, should be understood as meaning an object of length (L) and of diameter (D) such that (L) is very much greater than (D), wherein (D) is the diameter of the circle within which the cross section of the fiber is inscribed. For example, the ratio (L)/(D) (or form factor) may range from 3.5 to 2,500, such as from 5 to 500 and from 5 to 150. In one embodiment of the present disclosure, the fibers have a length ranging from 0.05 mm to 6 mm.

The non-expanded particles used in the present disclosure can have a mass per unit volume ranging from 500 kg/m³ to 5000 kg/m³, for instance from 900 kg/m³ to 3000 kg/m³ and from 900 kg/m³ to 2000 kg/m³.

The particles used according to the present disclosure can be colored or colorless.

Among the particles that may be used according to the present disclosure, non-limiting examples that may be mentioned include non-expanded microspheres of vinylidene chloride/acrylonitrile/methyl methacrylate copolymer, for instance those sold under the name “Expancel®” by the company Akzo Nobel under the references 820 DU 40 (10-16 μm) or 820 SL 40 (2-30 μm), and of acrylonitrile/methyl methacrylate, for instance those sold under the name “Expancel®” under the references 642 WU 40 (10-16 μm) or 051 DU 40 (9-15 μm). Further among the particles that can be used in the compositions according to the present disclosure, non-limiting mention may also be made of non-expanded microspheres of acrylonitrile homopolymer, for instance those sold under the name “Expancel 007WU®” (5-25 μm), “Micropearl F-series®” by the company Matsumoto or “Ucelite®” by the company UCB.

The particles sold under the name “Expancel®” as described above generally comprise in their cavity a compound in gaseous form.

In the compositions according to the present disclosure, the amount of compounds capable of swelling by the action of heat present in the compositions as disclosed herein can range from 0.1 % to 20%, for instance from 0.2% to 15% and such as from 0.5% to 10% by weight, relative to the total weight of the composition.

The cosmetically acceptable medium can be water or comprise water and at least one cosmetically acceptable solvent such as alcohols or water-solvent mixtures, for example, C₁-C₄ alcohols, such as ethanol and isopropanol. In one embodiment of the present disclosure, the alcohol is ethanol.

The composition according to the present disclosure can also comprise at least one additive chosen from nonionic, anionic, cationic, amphoteric and zwitterionic surfactants; fragrances; screening agents; preserving agents; proteins; vitamins; nonionic, anionic, cationic, amphoteric and zwitterionic polymers other than the fixing polymers described above; mineral, plant and synthetic oils; thickeners and any other additive conventionally used in cosmetic compositions, such as antidandruff agents, agents for combating hair loss, dyes, pigments, moisturizers such as glycerol and other polyols, and reducing agents.

These additives are optionally present in the composition in amount ranging from 0.001% to 20% by weight, relative to the total weight of the composition. The precise amount of each additive depends on its nature and may readily be determined by a person of ordinary skill in the art, and will depend on the hair application selected.

Needless to say, a person of ordinary skill in the art will take care to select the additive(s) such that the beneficial properties of the composition in accordance with the present disclosure are not, or are not substantially, adversely affected by the envisaged addition.

The compositions according to the present disclosure may be used for manufacturing numerous hair products such as, for example, products for fixing and/or holding the hair, conditioning products, or haircare products.

The compositions as disclosed herein can be packaged in various forms, for example, in pump-dispenser bottles or in aerosol cans, so as to be able to apply the composition in vaporized form or in mousse form. Such packaging forms are indicated, for example, when it is desired to obtain a spray, a lacquer or a mousse for fixing or treating the hair. The compositions in accordance with the present disclosure may also be in the form of creams, gels, emulsions, lotions or waxes.

When the composition according to the present disclosure is packaged in aerosol form in order to obtain a lacquer or a mousse, it comprises at least one propellant that may be chosen from volatile hydrocarbons such as n-butane, propane, isobutane and pentane, halogenated hydrocarbons, and mixtures thereof. Carbon dioxide, nitrous oxide, dimethyl ether (DME), nitrogen and compressed air may also be used as a propellant. Mixtures of propellants may also be used. In one embodiment of the present disclosure, dimethyl ether is used.

For example, the propellant can be present in an amount ranging from 5% to 90% by weight, such as from 10% to 60% by weight, relative to the total weight of the composition in the aerosol device.

Another aspect of the present disclosure is a cosmetic process for fixing and/or holding the hairstyle, comprising the application to the hair of a composition as disclosed herein, followed by the application of heat to the hair, at a temperature for example, ranging from 55° C. to 220° C., for instance from 65° C. to 220° C., for a period of time that can range from 2 seconds to 30 minutes.

Still another aspect of the present disclosure is the cosmetic use of the compositions described herein. For example, an aspect of the present disclosure is the use of compounds capable of swelling by the action of heat, for the preparation of compositions for holding and/or fixing the hairstyle.

Other than in the operating example, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific example are reported as precisely as possible. Any numerical value, however, inherently contain certain errors necessarily resulting from the standard deviation found in their respective testing measurements.

The following example is intended to illustrate the invention in a non-limiting manner.

### EXAMPLE 1

Composition A below was applied to a 5-gram lock of natural European chestnut-brown hair.

Composition A:

| | |
|---|---|
| Mexomer PW (1) | 6% AM |
| Expancel 820 Slurry (2) | 1% AM |
| Water | 85 g |
| Ethanol | 8 g |

AM: active materials
(1) vinyl acetate/vinyl p-tert-butylbenzoate/crotonic acid terpolymer 100% neutralized with 2-amino-2-methyl-1-propanol
(2) vinylidene/acrylonitrile/methyl methacrylate microspheres comprising isobutane

Composition A was applied using a pump-dispenser bottle onto natural hair. Composition A allowed good hairstyle shaping and hold to be obtained. The hair was then subjected to the action of heat of using a hairdryer at 105° C. for 20 seconds. The heat resulted in the irreversible expansion of the microspheres, which embrittled the welds between the hairs. The hair was thus easy to style and to disentangle.

1. A cosmetic process for fixing and/or holding a hairstyle, comprising
applying to hair a composition comprising, in a cosmetically acceptable medium, at least one fixing polymer and at least one compound capable of swelling by the action of heat,
followed by subjecting the hair to the action of heat, at a temperature ranging from 65° C. and 220° C.

2. The process according to claim 1, wherein the at least one fixing polymer is chosen from cationic, anionic, amphoteric and nonionic fixing polymers.

3. The process according to claim 2, wherein the at least one cationic fixing polymer is chosen from acrylic ester, methacrylic ester and amide homopolymers and copolymers comprising amine functional groups, cationic polysaccharides, quaternary copolymers of vinylpyrrolidone and of vinylimidazole, and chitosans.

4. The process according to claim 2, wherein the at least one anionic fixing polymer is chosen from acrylic and methacrylic acid homopolymers and copolymers and salts thereof, crotonic acid copolymers, C₄-C₈ monounsaturated carboxylic acid and anhydride copolymers, polyacrylamides comprising carboxylate groups, homopolymers and copolymers comprising sulfonic groups, anionic polyurethanes and anionic grafted silicone polymers.

5. The process according to claim 2, wherein the at least one amphoteric fixing polymer is chosen from copolymers comprising acidic vinyl units and basic vinyl units, crosslinked and acylated polyamino amides, polymers comprising zwitterionic units, chitosan-based polymers, modified (C₁-C₅)alkyl vinyl ether/maleic anhydride copolymers, amphoteric polyurethanes and amphoteric grafted silicone polymers.

6. The process according to claim 2, wherein the at least one nonionic fixing polymer is chosen from polyalkyloxazolines, vinyl acetate homopolymers and copolymers, acrylic ester homopolymers and copolymers, acrylonitrile copolymers, styrene homopolymers and copolymers, polyamides, vinyllactam homopolymers other than vinylpyrrolidone homopolymers, vinyllactam copolymers, nonionic polyurethanes and nonionic grafted silicone polymers.

7. The process according to claim 1, wherein the at least one fixing polymer is present in the composition in an amount ranging from 0.1% to 20% by weight, relative to the total weight of the composition.

8. The process according to claim 7, wherein the at least one fixing polymer is present in the composition in an amount ranging from 0.2% to 15% by weight, relative to the total weight of the composition.

9. The process according to claim 8, wherein the at least one fixing polymer is present in the composition in an amount ranging from 0.5% to 10% by weight, relative to the total weight of the composition.

10. The process according to claim 1, wherein the at least one compound capable of swelling by the action of heat is in the form of heat-expandable particles.

11. The process according to claim 10, wherein the heat-expandable particles are capable of expanding at a temperature of greater than or equal to 45° C.

12. The process according to claim 11, wherein the heat-expandable particles are capable of expanding at a temperature of greater than or equal to 50° C.

13. The process according to claim 12, wherein the heat-expandable particles are capable of expanding at a temperature of greater than or equal to 70° C.

14. The process according to claim 13, wherein the heat-expandable particles are capable of expanding at a temperature of greater than or equal to 80° C.

15. The process according to claim 14, wherein the heat-expandable particles are capable of expanding at a temperature greater than or equal to 90° C.

16. The process according to claim 15, wherein the heat-expandable particles are capable of expanding at a temperature greater than or equal to 100° C.

17. The process according to claim 10, wherein the heat-expandable particles are thermoplastic.

18. The process according to claim 10, wherein the heat-expandable particles are hollow particles comprising a continuous envelope and at least one cavity.

19. The process according to claim 18, wherein the envelope comprises at least one polymer resulting from the polymerization of monomers chosen from methacrylic and acrylic acid esters, vinylidene chloride, acrylonitrile, styrene and derivatives thereof, butadiene and derivatives thereof, and mixtures thereof.

20. The process according to claim 19, wherein the at least one polymer is chosen from polymers comprising at least one monomeric unit chosen from methyl acrylate and methacrylate derivatives, polymers comprising at least one monomeric unit chosen from acrylonitrile derivatives, polymers comprising at least one monomeric unit chosen from acrylonitrile and methyl methacrylate derivatives, polymers comprising at least one monomeric unit chosen from styrene and acrylonitrile derivatives, polymers comprising at least one monomeric unit chosen from vinylidene chloride and acrylonitrile derivatives, and polymers comprising at least one monomeric unit chosen from vinylidene chloride and vinyl chloride derivatives.

21. The process according to claim 20, wherein the at least one polymer is chosen from vinylidene chloride/acrylonitrile/methyl methacrylate polymers, acrylonitrile/methyl methacrylate polymers and acrylonitrile homopolymers.

22. The process according to claim 18, wherein the at least one cavity comprises at least one compound capable of exhibiting, in response to heating to a temperature ranging from 45° C. to 200° C. and at a substantially constant pressure, an increase by at least a factor of 30 of its volume relative to its volume at room temperature.

23. The process according to claim 22, wherein the at least one compound is either in gaseous or liquid form.

24. The process according to claim 23, wherein the at least one gaseous compound is chosen from air, nitrogenand hydrocarbons comprising from 1 to 4 carbon atoms

25. The process according to claim 23, wherein the at least one liquid compound has a vaporization temperature ranging from 45° C. to 200° C.

26. The process according to claim 25, wherein the at least one liquid compound has a vaporization temperature ranging from 80° C. to 200° C.

27. The process according to claim 25, wherein the at least one liquid compound is chosen from hydrocarbons comprising from 5 to 15 carbon atoms.

28. The process according to claim 10, wherein the heat-expandable particles are globular or elongated.

29. The process according to claim 28, wherein the heat-expandable particles are spherical and have a particle size, expressed as the weight-average “effective” diameter, ranging from 0.5 μm to 200 μm.

30. The process according to claim 29, wherein the heat-expandable particles are spherical and have a particle size, expressed as the weight-average “effective” diameter, ranging from 1 μm to 100 μm.

31. The process according to claim 30, wherein the heat-expandable particles are spherical and have a particle size, expressed as the weight-average “effective” diameter, ranging from 4 μm to 50 μm.

32. The process according to claim 31, wherein the heat-expandable particles are spherical and have a particle size, expressed as the weight-average “effective” diameter, ranging from 5 μm to 40 μm.

33. The process according to claim 10, wherein the heat-expandable particles are fibers.

34. The process according to claim 33, wherein the fibers have a length ranging from 0.05 mm to 6 mm.

35. The process according to claim 10, wherein the heat-expandable particles have a mass per unit volume ranging from 500 kg/m³ to 5000 kg/m³.

36. The process according to claim 35, wherein the heat-expandable particles have a mass per unit volume ranging from 900 kg/m³ to 3000 kg/m³.

37. The process according to claim 36, wherein the heat-expandable particles have a mass per unit volume ranging from 900 kg/m³ to 2000 kg/m³

38. The process according to claim 1, wherein the at least one compound capable of swelling by the action of heat is present in an amount ranging from 0.1% to 20% by weight, relative to the total weight of the composition.

39. The process according to claim 38, wherein the at least one compound capable of swelling by the action of heat is present in an amount ranging from 0.2% to 15% by weight, relative to the total weight of the composition.

40. The process according to claim 39, wherein the at least one compound capable of swelling by the action of heat is present in an amount ranging from 0.5% to 10% by weight, relative to the total weight of the composition.

41. The process according to claim 1, wherein the composition further comprises at least one additive chosen from nonionic, anionic, cationic, amphoteric and zwitterionic surfactants; fragrances; screening agents; preserving agents; proteins; vitamins; nonionic, anionic, cationic and zwitterionic polymers other than the fixing polymers present in the composition; mineral, plant and synthetic oils; thickening agents; antidandruff agents; agents for combating hair loss; dyes; pigments; moisturizers and reducing agents.

42. A cosmetic hair composition comprising, in a cosmetically acceptable medium, at least one fixing polymer and at least one compound capable of swelling by the action of heat, wherein said at least one compound is in the form of fibers.

43. The composition according to claim 42, wherein the fibers have a length ranging from 0.05 mm to 6 mm.
